(19) United States
(12) Patent Application Publication
(10) Pub. No.: US 2026/0241147 A1
(43) Pub. Date: Aug. 20, 2026

(54) Medical Apparatus for Enhanced Endoluminal Delivery of Treatment Agent

(71) Applicant: Bard Peripheral Vascular, Inc., Franklin Lakes, NJ (US)

(72) Inventors: Peng Guo, Shanghai (CN); John Ong, Singapore (SG); Xiaotian Yu, Jiangsu (CN); Yang Zheng, Shanghai (CN); Hely Li, Shanghai (CN); Jiayi Chai, Shanghai (CN); Jiahuan Wan, Shanghai (CN)

(21) Appl. No.: 19/055,789

(22) Filed: Feb. 18, 2025

(51) Int. Cl. A61M 25/10; A61M 37/00

(52) U.S. Cl. CPC A61M 25/1011; A61M 37/0092; A61M 2025/105; A61M 2025/1052; A61M 2025/1079; A61M 2025/1086

(57) ABSTRACT

A medical apparatus is for delivering a treatment agent, such as a cancer drug associated with a chemotherapy regimen, to an endoluminal space adjacent to the cancer. The apparatus includes a shaft supporting a first inflatable balloon. The first inflatable balloon or the catheter shaft is adapted for delivering the treatment agent to the endoluminal space. An energy source is also provided for diffusing the treatment agent. A second inflatable balloon may also be provided which may be received in an external channel of the first inflatable balloon. The catheter shaft may also include a first port for introducing the treatment agent to a chamber formed in the endoluminal space by the first and second balloons once inflated, as well as a second port for introducing energy from the energy source to this chamber to enhance the penetration and diffusion of the treatment agent.

### BACKGROUND

A variety of medical apparatuses for use in delivering treatment to a patient in need of such treatment are well known. In one particular form, a balloon catheter may be used to deliver a treatment agent, such as a drug or other pharmaceutical composition, endoluminally, such as in blood vessels forming the vasculature. An important aspect of achieving a successful treatment outcome is the ability to control the delivery of the treatment agent to target precisely a particular location and also ensure efficacy as a result of the targeted delivery, which heretofore have both been lacking using known techniques.

One particular medical condition that may benefit from such enhanced treatment agent delivery is pancreatic cancer, which presently is considered fourth in the list of fatal cancers. The treatment of locally advanced pancreatic cancer (LAPC) is extremely difficult, primarily due to the presence of a complicated tumor microenvironment (TME). An exemplary TME is illustrated in FIG. 1, which illustrates the presence of a variety of tumors associated with the pancreas P and adjacent gall bladder G, such as for example a neuroendocrine tumor N, a cholangiocarcinoma C, or an adenocarcinoma A. The pancreas P is also adjacent to other organs or parts thereof, such as the duodenum D, further complicating treatment delivery anatomically. The surrounding anatomy is also such that precise control is required to deliver any treatment agent to a targeted location or tumor to achieve an effective result, as widespread distribution of the treatment may result in incidental and deleterious damage to healthy tissues and/or organs.

Accordingly, a need is identified for a medical apparatus for use in the endoluminal delivery of a treatment agent in a highly controlled, predictable and effective manner. The apparatus would be intended to target a variety of tumors associated with the pancreas with a treatment agent. In particular, the treatment could be regulated to precise locations by introducing a balloon adapted for delivering the treatment agent via parts of the adjacent vasculature, such as the portal vein PV or the superior mesenteric vein SMV and artery SMA. Overall, a better outcome in terms of treatment could be achieved using such an approach, without any significant enhancement of the cost or increase in the complexity of the treatment regimen.

### SUMMARY

An object of the disclosure is to provide a medical apparatus for use in the endoluminal delivery of a treatment agent in a highly controlled, predictable and effective manner. The apparatus may target a variety of tumors, such as those associated with the pancreas, with a treatment agent. As a result of the design of the apparatus, the treatment may be regulated to precise locations by introducing a balloon adapted for delivering the treatment agent in a directed fashion. Overall, a better outcome in terms of treatment may be achieved using such an approach, without any significant enhancement of the cost or increase in complexity of the treatment regimen.

According to a first aspect of the disclosure, a medical apparatus for delivering a treatment agent to an endoluminal space includes a catheter shaft. A first inflatable balloon is supported by the catheter shaft. The first inflatable balloon or the catheter shaft is adapted for delivering the treatment agent to the endoluminal space. At least one energy source is provided for diffusing the treatment agent delivered from the shaft or the first inflatable balloon.

In this or other embodiments, the first inflatable balloon includes at least one external channel having at least one delivery port corresponding to the at least one energy source. The at least one external channel may comprise a plurality of external channels adapted for delivering the treatment agent. Each external channel may include a plurality of delivery ports. The at least one energy source may comprise a plurality of energy sources, each corresponding to one delivery port of the plurality of delivery ports.

In these or other embodiments, a second inflatable balloon is supported by the shaft. The second inflatable balloon includes at least one external channel. The first inflatable balloon is at least partially received in the at least one external channel of the first inflatable balloon. The first inflatable balloon may be adapted for delivering the treatment agent through micropores. One or both of the first and second inflatable balloons comprise one or more radiopaque markings to facilitate orientation.

In some embodiments, the at least one energy source comprises at least one ultrasonic generator. The at least one ultrasonic generator may be attached to the shaft within the balloon. The at least one energy source may comprise a plurality of ultrasonic generators, each comprising a band.

In these or other embodiments, the catheter shaft includes a first port for introducing the treatment agent to the endoluminal space and a second port for transmitting energy from the at least one energy source to the endoluminal space. The at least one energy source may comprise an ultrasonic generator attached to a wire insertable into the shaft to position the ultrasonic generator at or adjacent to the second port. The first inflatable balloon may be proximal of the first port and the second port, and further including a second inflatable balloon distal of the first port and the second port.

According to a further aspect of the disclosure, a medical apparatus for delivering a treatment agent to an endoluminal space is provided. The apparatus includes a catheter shaft and a first inflatable balloon supported by the catheter shaft. The first inflatable balloon is adapted to deliver the treatment agent. A second inflatable balloon also supported by the catheter shaft includes an external channel for at least partially receiving the first inflatable balloon. In some embodiments, the first inflatable balloon is adapted for delivering the treatment agent through micropores, and further including at least one energy source for diffusing the treatment agent via the micropores.

According to yet another aspect of the disclosure, a medical apparatus for delivering a treatment agent to an endoluminal space includes a catheter shaft and a first inflatable balloon supported thereby. The first inflatable balloon includes at least one external channel having at least one delivery port. At least one energy source is provided for diffusing the treatment agent from the at least one delivery port.

In some embodiments, the at least one external channel comprises a plurality of external channels, each having at least one delivery port adapted for delivering the treatment agent. Each external channel of the plurality of external channels may include a plurality of delivery ports. The at least one energy source may comprise a plurality of energy sources, each corresponding to one delivery port of the plurality of delivery ports. The at least one energy source may be connected to the shaft adjacent to the at least one delivery port.

### BRIEF DESCRIPTION OF THE DRAWINGS

The above and further advantages of the disclosure may be better understood by referring to the following description in conjunction with the accompanying drawings in which:

FIG. 1 illustrates an exemplary portion of the human anatomy that may constitute an environment of use for the disclosed medical apparatuses.

FIG. 2 is a perspective view of a balloon catheter.

FIG. 3 is a cutaway rear perspective view of the balloon portion of the balloon catheter of FIG. 2.

FIG. 3A is a cross-sectional view taken along line 3A-3A of FIG. 3.

FIG. 4 is an alternative embodiment of a balloon catheter.

FIG. 5 is one embodiment of an apparatus for the enhanced endoluminal delivery of a treatment agent according to one aspect of the disclosure.

FIG. 5A is a cross-sectional view taken along line 5A-5A of FIG. 5.

FIGS. 5B, 5C, and 5D are side views of the apparatus shown in FIG. 5 in various orientations.

FIG. 6 is another embodiment of an apparatus for the enhanced endoluminal delivery of a treatment agent according to one aspect of the disclosure.

FIG. 6A is a cross-sectional view taken along line 6A-6A of FIG. 6.

FIG. 7 is yet another embodiment of an apparatus for the enhanced endoluminal delivery of a treatment agent according to one aspect of the disclosure.

FIG. 8 is a partially cutaway side view of an ultrasonic guidewire.

FIG. 9. Is a side view of the embodiment of FIG. 7.

FIG. 9A is a cross-sectional view taken along line 9A-9A of FIG. 9

FIGS. 10 and 10A illustrate the apparatus of FIG. 7 in various states of use.

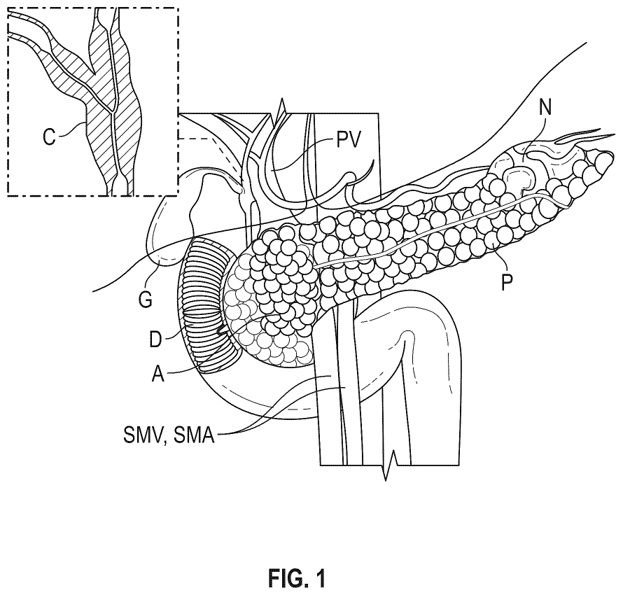

The dimensions of some of the elements in the drawings may be exaggerated relative to other elements for clarity or several physical components may be included in one or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the disclosure in relation to various inventions. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination. The description provided below and in regard to the figures applies to all embodiments unless noted otherwise, and features common to each embodiment are similarly shown and numbered to the greatest extent possible to lend clarity, but should not be considered limiting in any respect.

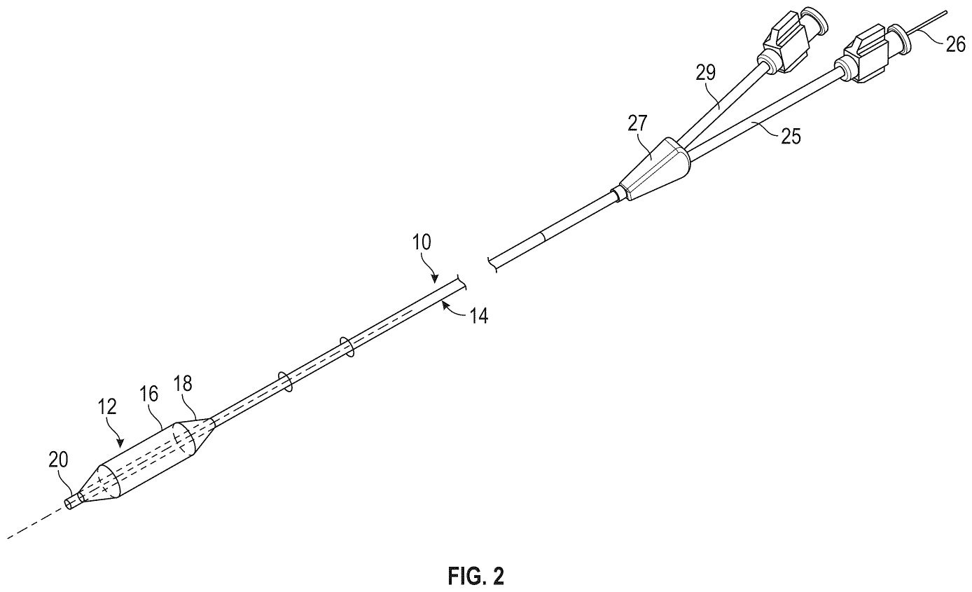

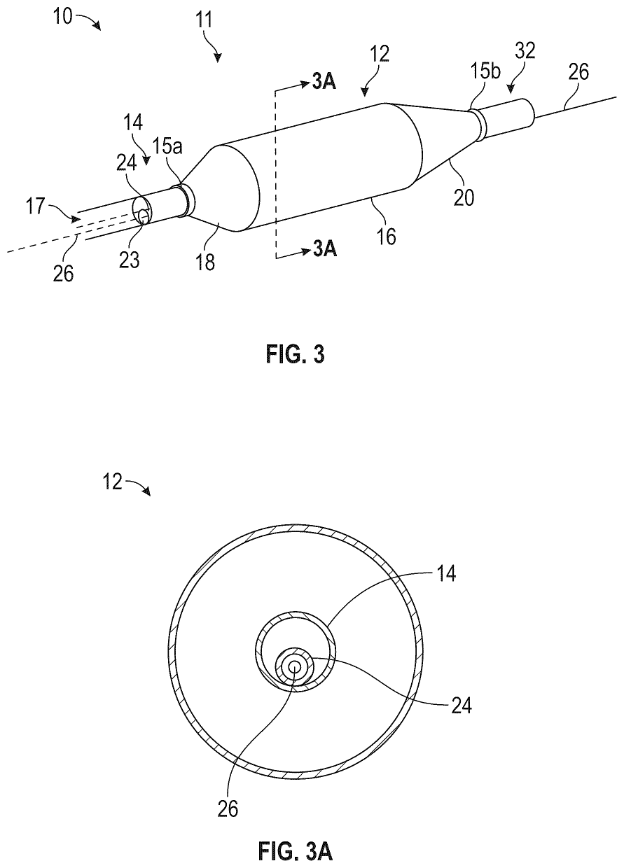

With reference to FIGS. 2, 3, and 3A, provided is a catheter 10 having a distal end portion 11 with a balloon 12 mounted on a catheter shaft 14. The balloon 12 has an intermediate portion 16, or “barrel,” and end portions 18, 20. In one embodiment, the end portions 18, 20 reduce in diameter to join the intermediate portion 16 to the catheter shaft 14 (and thus portions 18, 20 are generally termed cones or cone portions). The balloon 12 is sealed at balloon ends (proximal end 15a and distal end 15b) on the cone portions 18, 20 to allow the inflation of the balloon 12 via one or more inflation lumens 17 extending within catheter shaft 14 and communicating with the interior of the balloon 12.

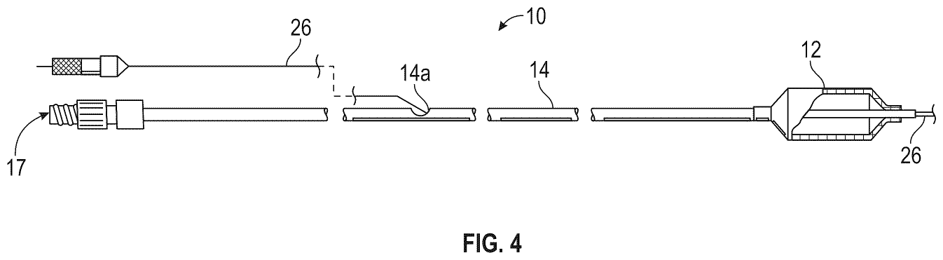

The catheter shaft 14 may also include an elongated, tubular shaft 24 forming a guidewire lumen 23 that directs the guidewire 26 through the catheter 10, and along the distal end portion of which the balloon 12 may be located. As illustrated in FIG. 3, this guidewire 26 may extend through the proximal end portion of the catheter 10 and a first port 25 of a connector or hub 27 into the lumen 23 to achieve an “over the wire” (OTW) arrangement, but could also be provided in a “rapid exchange” (RX) configuration (in which the guidewire 26 exits a lateral opening 14a closer to the distal end; see FIG. 4) or else is fed through the tip at a passage distally of the balloon 12 (“short” RX; not shown). A second port 29 may also be associated with catheter 10, such as by way of connector 27, for introducing a fluid (e.g., saline, a contrast agent, or both) into the interior compartment of the balloon 12 via the inflation lumen 17.

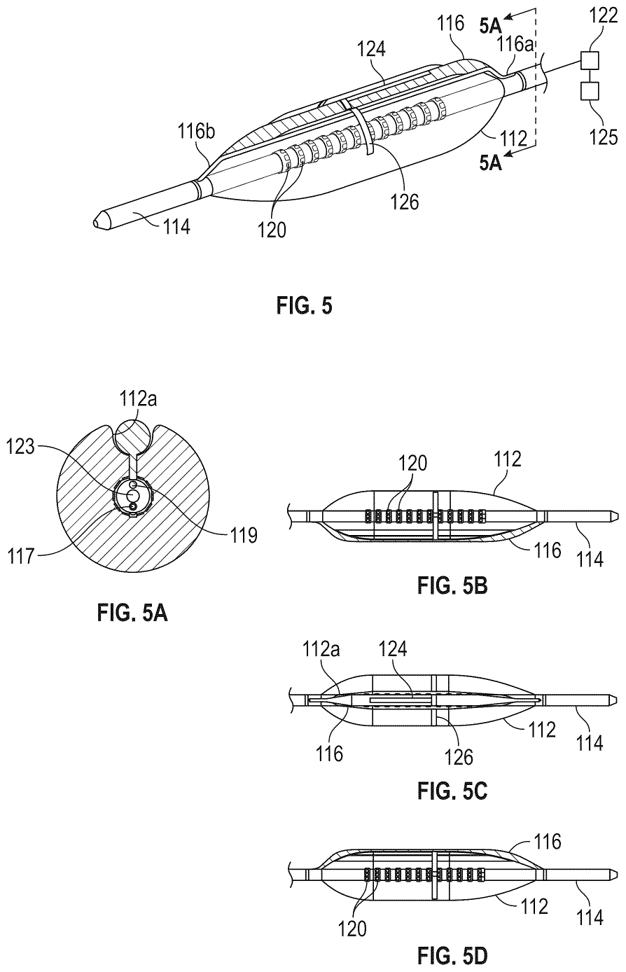

According to a first aspect of the disclosure, an apparatus 100 is provided for the enhanced endoluminal delivery of a treatment agent. As shown in FIG. 5, the apparatus 100 comprises a catheter shaft 114 supporting a first inflatable balloon 112. As perhaps best understood from the partially cross-sectional view of FIG. 5A, the shaft 114 includes a first inflation lumen 117 in communication with an interior compartment of the first inflatable balloon 112, such as via a port, as well as a guidewire lumen 123.

As can be also understood from FIG. 5A, the first inflatable balloon 112 includes an external channel 112a. Received in this channel 112a is a second inflatable balloon 116, which is also in communication with a second inflation lumen 119 in the shaft 114, such as at a proximal end 116a, and sealed to the shaft at the distal end 116b thereof. This second balloon 116 is thus considered nested substantially within the channel 112a, and may extend the entire length of the first balloon 112, but this is not a requirement. As can be appreciated, the second balloon 116 is substantially smaller in diameter than the first balloon 112 (as illustrated, approximately 5-6 times smaller, but the relative proportions may be varied). The width and depth of the channel 112a is at least as great as the diameter of the second balloon 116, and as can be appreciated, the first balloon 112 surrounds the second balloon 116 on all sides except for an exposed face. The balloons 112, 116 may be discrete, may be connected together (such as by an adhesive (or not), or may be a single unitary structure.

The second balloon 116 may be formed of a microporous material for purposes of allowing for delivery of a treatment agent introduced thereto. For purposes of this disclosure may be considered as is a material containing pores with diameters of less than about 10 nanometers and, more specifically, less than about two nanometers. Examples of microporous materials include conjugated microporous polymers (CMPs), zeolites, metal-organic frameworks, and covalent organic frameworks. The particular manner and degree of porosity achieved by the selected material is not considered essential, and may vary depending on the particular application of the apparatus 100.

The shaft 114 supporting the balloons 112, 116 may also be provided with one or more energy sources. In the example shown, these energy sources may comprise a plurality of spaced bands 120, each adapted for generating energy in the form of ultrasound waves. The bands 120 may be connected to a remote source of power 122 associated with the shaft 114, and via an associated controller 125 may be controlled to generate ultrasound waves having a frequency of from about 20 kHz to about 1 MHz, and which bands could all generate the same frequency or a multiple frequency set.

With continued reference to FIG. 5, as well as the various side views of FIGS. 5B, 5C, and 5D, it can be understood that one or both of the balloons 112, 116 may be provided with indicators that are visible at the endoluminal treatment location, such as under fluoroscopy or digital subtraction angiography (DSA). For instance, a first radiopaque marking 124 may extend transversely along the first balloon 112, and a second radiopaque marking 126 may extend longitudinally along the second balloon 116.

As can be appreciated by comparing FIGS. 5B, 5C, and 5D, these markings 124, 126 when viewed under imaging external to the vessel in which the balloons 112, 116 are positioned allow the clinician to assess the relative orientation. This may be useful ensure that the portion of the second balloon 116 exposed by the channel 112a is facing the correct direction to achieve the desired treatment effect. Similar indicators may also be provided on the shaft 114, if desired.

In use, the catheter 114 including the deflated balloons 112, 116 may be delivered to the desired vessel depending on a desired location for treatment, such as the position of an adjacent tumor. Contrast may then be injected to the second balloon 116, and under fluoroscopy, the radiopaque indicators may be used to assist in judging the relative orientation. The catheter 114 may be rotated as necessary to ensure that the open face of the channel 112a and thus the second balloon 112 faces in the desired direction, such as towards the targeted tumor.

Once the proper positioning is confirmed, the second balloon 116 may be flushed using a suitable fluid (e.g., saline). The first balloon 112 may then be inflated to an appropriate pressure with a suitable fluid (e.g., saline). The energy source(s) such as bands 120 may then be activated, and a treatment agent injected into the second balloon 116, which may be done at a preset flow rate.

The energy wave created as a result of the activation of the bands 120 both improves the permeability of the adjacent tissues of the vessel and also enhances the diffusion speed of the treatment agent. The arrangement of balloons 114, 116 also ensures that the treatment agent is delivered primarily in the targeted direction, and in a limited range (e.g., one that is substantially less than 360 degrees, and more in a range of from 0-180 degrees including each possible value in that range; more particularly, less than 90 degrees including each value in that range; and even more particularly, less than 45 degrees including each value in that range). This targeted approach tends to reduce or avoid the hazard to healthy tissues adjacent to the desired treatment location and improve the concentration of the treatment agent only at that location.

Once the treatment regimen is completed, the power may be turned off to halt the diffusion of the treatment agent. The balloons 112, 116 may be deflated, repositioned, and re-deployed to treat a different location. Alternatively, the catheter shaft 114 may be withdrawn from the endoluminal space.

Without wishing to be bound to a particular scientific theory, the improved effect is believed to result from the acoustic cavitation created by an ultrasound-induced mechanical wave, which creates microbubbles that temporarily open the endothelial junctions in the targeted tissue. This phenomenon occurs when the ultrasound wave propagates through the tissue, causing the formation of microbubbles in the blood. These microbubbles oscillate and grow, ultimately undergoing violent collapse. The resulting shear forces and micro-streaming effects temporarily disrupt the endothelial junctions, creating transient openings in the vessel walls. This disruption allows therapeutic agents to penetrate more deeply into the vascular tissues and surrounding areas, thereby increasing the efficacy of the delivery of the treatment agent.

The thermal effect of ultrasound waves also leads to localized hyperthermia, which in turn enhances blood flow and increases vascular permeability. This thermal-induced hyperthermia can soften the vascular tissues, making the endothelial junctions more pliable and thereby facilitating the penetration of therapeutic agents. The elevated temperature can further promote the diffusion of treatment agents or drugs across the cellular membranes, allowing for more effective delivery to targeted sites in the vasculature. The acoustical and thermal effects induced by intravascular ultrasound waves are thus capable of enhancing vascular and cellular membrane permeability, allowing delivered treatment agent to extravagate into the surrounding region.

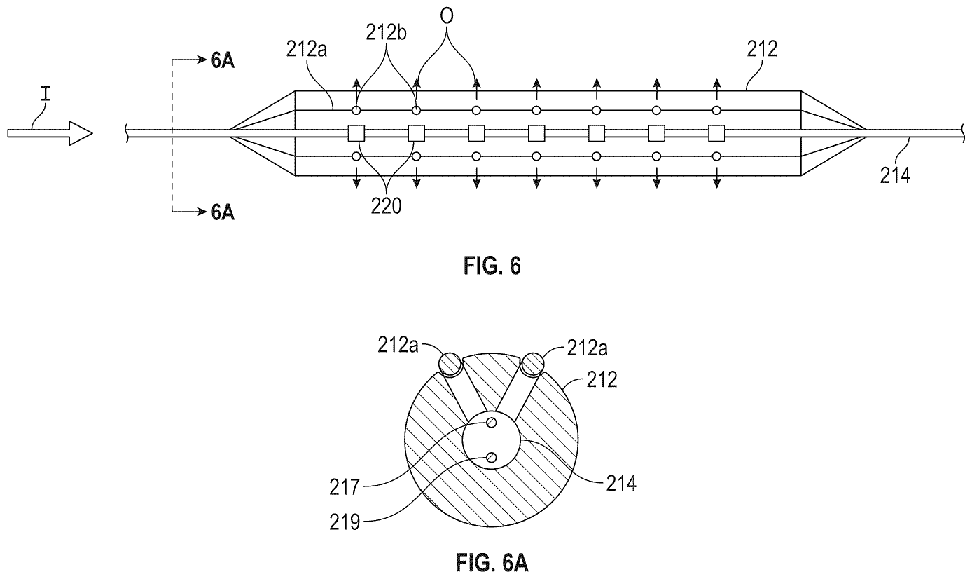

A further embodiment of a medical apparatus 200 for the enhanced endoluminal delivery of a treatment agent leveraging the disclosed principles of operation is shown schematically in FIGS. 6 and 6A. In this version, an inflatable balloon 212 supported by a shaft 214 includes one or more external channels 212a, which may be spaced apart in a circumferential direction and extend along a particular portion of the balloon less than the full circumference (e.g., 0-180 degrees).

Each external channel 212a include one or more openings. These openings thus serve as delivery ports 212b for the treatment agent when introduced to the channel 212a. These ports 212b may in turn be associated or aligned with corresponding energy sources 220 on the shaft 214, which may be in the form of bands as previously noted, or may take other forms. Each channel 212a may be independent of the other, or may cooperate with other channels. This arrangement also need not use radiopaque markers or the like, since the channels 212a may be provided with contrast agent in use to determine the correct orientation.

In use, upon proper positioning and inflation of the balloon 212, such as via a lumen 217 in the shaft 114 for receiving an inflation fluid and another lumen (not shown) for receiving a guidewire for guidance to a desired location, a treatment agent may be introduced to the channels 212a from an external source. This introduction may be achieved via one or more lumens 219 in the shaft 214 associated with the channels 212a (note arrow I). When combined with the generation of energy by the sources 220, the treatment agent is caused to diffuse from the ports 212b associated with the channels 212a (arrows O). Repositioning or withdrawal of the apparatus 200 may be done as necessary or desired.

As can be appreciated, similar benefits to those described above in terms of improving the permeability of the adjacent tissues of the vessel and also enhancing the diffusion speed of the treatment agent. The channels 212a may also be provided along only one side of the balloon 212, thus allowing for the desired directional control of diffusion of the treatment agent toward a diseased tissue or one otherwise in need of treatment.

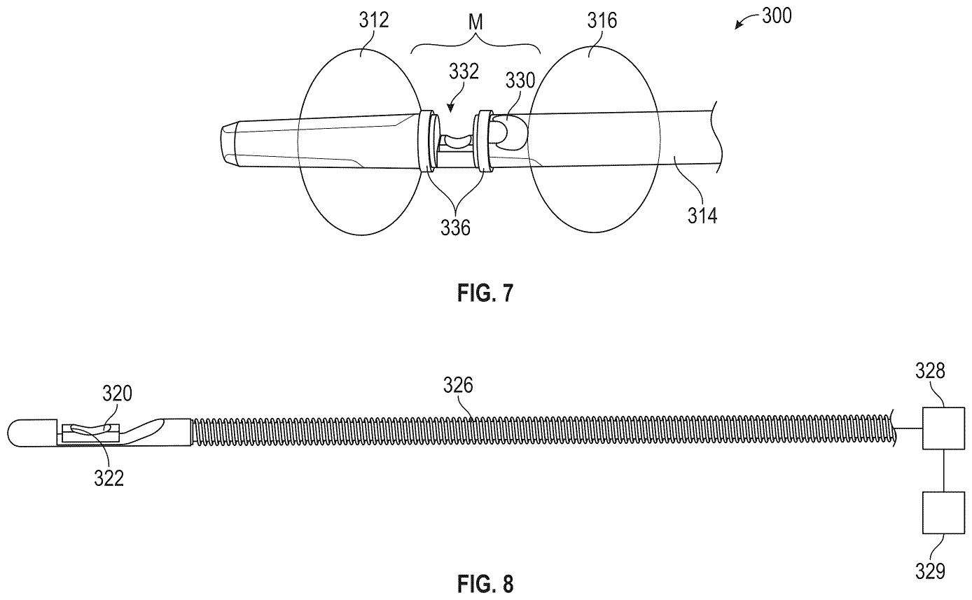

A further embodiment of a medical apparatus 300 for the enhanced endoluminal delivery of a treatment agent further leveraging the disclosed principles of operation is described with reference to FIGS. 7, 8, 9, 9A, 10 and 10A. Referring first to FIG. 7, a catheter shaft 314 supports proximal and distally positioned inflatable balloons 312, 316. With added reference to FIG. 8, a portion 314a of the shaft 314 between the balloons 312, 316 includes a first port 330 for introducing a treatment agent, and a second port 332 for introducing energy transmitted thereto from an energy source. These ports 330, 332 may be spaced apart in a relatively close relationship, and may generally be provided on the same side of the shaft 314, but could be on different sides since the treatment agent will disperse quickly into surrounding areas. In any case, the ports 330, 332 are considered to be provided within the confined space created by the balloons 312, 316 once inflated or expanded.

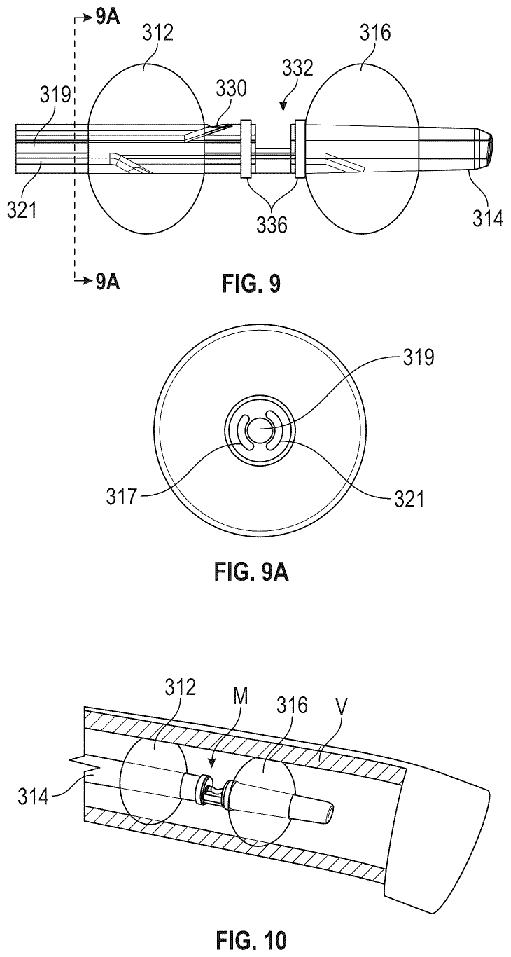

As shown in FIG. 9, the energy source may be selectively associated with the shaft 314. In one particular form, the source comprises an ultrasonic generator 320 connected adjacent to a distal end of a guidewire 326 near the tip thereof. The guidewire 326 may serve to supply power to the associated generator 320 from an external or internal source 328, which may be controlled by a controller 329. The generator 320 may be mounted on or form part of a housing 322 connected to the wire 326 in such a manner as to maintain a consistent outer diameter thereof, and thus allow for smooth introduction and removal. The wire 326 may comprise a coiled shaft to enhance flexibility for purposes of traversing tortuous anatomy.

With reference to FIG. 9A, which is a cross-sectional view taken along line 9A-9A of FIG. 9, a first lumen 317 may be provided in the shaft 314 in communication with the first port 330. A second lumen 319 may also be provided for receiving the guidewire 326 and guiding the distal end to locate the ultrasonic generator 320 adjacent to the second port 332. A third lumen 321 (which may be divided into two generally symmetrical lumens) may also be provided for supplying inflation fluid to inflate the balloons 312, 316.

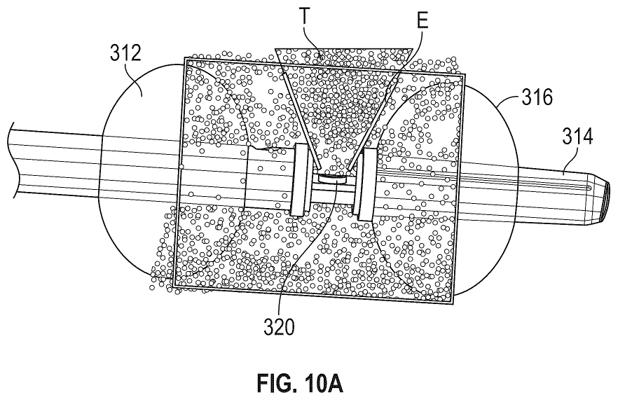

In use, and with reference to FIGS. 10 and 10A, the catheter shaft 314 may be positioned at the desired endoluminal treatment location, such as within a vessel V, using a conventional guidewire (not shown). The catheter shaft 314 may be oriented such that the ports 330, 332 are directed toward or target the same treatment location. Proper positioning may be aided by the use of indicators visible from outside the body during the procedure (such as via fluoroscopy; note for example radiopaque rings 336 associated with the shaft 314).

Once in the proper position, the balloons 312, 316 may be simultaneously inflated to create a compartment M therebetween. The treatment agent T may then be introduced to the infusion lumen 321 and exit via port 330, as shown in FIG. 10A. Energy E such as in the form of an ultrasonic wave applied via the generator 320 to the treatment agent T captured in the chamber formed by the inflated balloons 312, 316 via port 332, which may be considered a sonophoretic window. The combined action of the treatment agent and the applied energy thus achieves the beneficial enhanced permeability and diffusion, as previously described, in a highly targeted, controlled, and repeatable manner, and without significant impact to surrounding tissues. Repositioning or withdrawal of the apparatus 300 may be done as necessary or desired.

As used herein, the following terms have the following meanings: “A”, “an”, and “the” as used herein refers to both singular and plural referents unless the context clearly dictates otherwise. By way of example, “a compartment” refers to one or more than one compartment. “About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which such modifiers refer is itself also specifically disclosed. Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A medical apparatus for delivering a treatment agent to an endoluminal space, comprising:
a catheter shaft;
a first inflatable balloon supported by the catheter shaft, the first inflatable balloon or the catheter shaft adapted for delivering the treatment agent to the endoluminal space; and
at least one energy source for diffusing the treatment agent in the endoluminal space.

2. The medical apparatus of claim 1, wherein the first inflatable balloon includes at least one external channel having at least one delivery port corresponding to the at least one energy source.

3. The medical apparatus of claim 2, wherein at least one external channel comprises a plurality of external channels adapted for delivering the treatment agent, each channel including a plurality of delivery ports, and wherein the at least one energy source comprises a plurality of energy sources, each corresponding to one delivery port of the plurality of delivery ports.

4. The medical apparatus of claim 1, further including a second inflatable balloon supported by the catheter shaft.

5. The medical apparatus of claim 4, wherein the second inflatable balloon includes at least one external channel, and the first inflatable balloon is at least partially received in the at least one external channel of the first inflatable balloon.

6. The medical apparatus of claim 5, wherein the first inflatable balloon is adapted for delivering the treatment agent via micropores.

7. The medical apparatus of claim 5, wherein one or both of the first and second inflatable balloons comprise one or more radiopaque markings to facilitate orientation.

8. The medical apparatus of claim 1, wherein the at least one energy source comprises at least one ultrasonic generator.

9. The medical apparatus of claim 8, wherein the at least one ultrasonic generator is attached to the catheter shaft.

10. The medical apparatus of claim 8, further comprising a plurality of bands attached to the shaft, and the at least one energy source comprises a plurality of ultrasonic generators, each connected to one band of the plurality of bands.

11. The medical apparatus of claim 1, wherein the shaft includes a first port for introducing the treatment agent to the endoluminal space and a second port for transmitting energy from the at least one energy source to the endoluminal space.

12. The medical apparatus of claim 11, wherein the at least one energy source comprises an ultrasonic generator attached to a wire insertable into the catheter shaft to position the ultrasonic generator at or adjacent to the second port.

13. The medical apparatus of claim 11, wherein the first inflatable balloon is proximal of the first port and the second port, and further including a second inflatable balloon distal of the first port and the second port.

14. A medical apparatus for delivering a treatment agent to an endoluminal space, comprising:
a catheter shaft;
a first inflatable balloon supported by the catheter shaft and adapted to deliver the treatment agent; and
a second inflatable balloon supported by the catheter shaft and having an external channel for at least partially receiving the first inflatable balloon.

15. The medical apparatus of claim 14, wherein the first inflatable balloon is adapted for delivering the treatment agent via micropores, and further including at least one energy source for diffusing the treatment agent.

16. The medical apparatus of claim 15, wherein the at least one energy source comprises an ultrasonic generator attached to the catheter shaft.

17. A medical apparatus for delivering a treatment agent to an endoluminal space, comprising:
a catheter shaft;
a first inflatable balloon supported by the catheter shaft, the first inflatable balloon including at least one external channel having at least one delivery port; and
at least one energy source for diffusing the treatment agent from the at least one delivery port.

18. The medical apparatus of claim 17, wherein at least one external channel comprises a plurality of external channels, each having at least one delivery port adapted for delivering the treatment agent.

19. The medical apparatus of claim 18, wherein each external channel of the plurality of external channels includes a plurality of delivery ports, and wherein the at least one energy source comprises a plurality of energy sources, each corresponding to one delivery port of the plurality of delivery ports.

20. The medical apparatus of claim 17, wherein the at least one energy source is connected to the shaft adjacent to the at least one delivery port.
